(19) United States
(12) Patent Application Publication
(10) Pub. No.: US 2025/0046401 A1
(43) Pub. Date: Feb. 6, 2025

(54) BASIN-WISE CONCENTRATION PREDICTION

(71) Applicant: TOTALENERGIES ONETECH, Courbevoie (FR)

(72) Inventors: Axel PORLAN, Courbevoie (FR); Frank DESPINOIS, Courbevoie (FR)

(21) Appl. No.: 18/717,903

(22) PCT Filed: Dec. 7, 2021
(86) PCT No.: PCT/IB2021/000845

§ 371 Date: Jun. 7, 2024

(51) Int. Cl. G16C 20/30; G01N 33/20; G16C 20/70

(52) U.S. Cl. CPC G16C 20/30; G01N 33/20; G16C 20/70

(57) ABSTRACT

It is hereby proposed a computer-implemented method of machine-learning a plurality of predictive basin-wise models. Each predictive basin-wise model is configured for predicting a concentration of an element at a given location in a saline aquifer of a respective basin. The machine-learning method comprises, for each basin and with respect to a predetermined set of one or more geochemical variables, providing a dataset, and learning the predictive basin-wise model based on the dataset. The dataset comprises, for respective saline aquifer locations of the basin, training samples. Each training sample includes a measurement of one or more geochemical variables of the predetermined set. Each training sample further includes a respective ground truth value. The ground truth value represents a concentration of the element at the respective saline aquifer location. Such a method forms an improved solution for analysis of a saline aquifer with respect to a given element of interest.

### TECHNICAL FIELD

The disclosure relates to the field of computer programs and systems, and more specifically to computer-implemented methods, data structures and devices for predicting a concentration of an element at a given location in a saline aquifer of a respective basin, and to a process that includes such prediction for storing CO2 in a saline aquifer of a respective basin.

### BACKGROUND

Saline aquifers are geological formations consisting of water permeable rocks that are saturated with salt water, called brine. Such water is rich in various metals and minerals and is thereby unsuitable for consumption or being discharged directly in the environment. Withdrawing water at a location of a saline aquifer thus requires heavy water treatment processes. To balance the costs of such treatment, some actors are considering recovering valuable elements contained in the withdrawn water, in particular metals such as lithium.

Within this context, there is a need for improved solutions notably for recovering elements of interest in a saline aquifer.

### SUMMARY

It is therefore provided a computer-implemented method of machine-learning a plurality of predictive basin-wise models, referred hereinafter as to the “machine-learning method”. Each predictive basin-wise model is configured for predicting a concentration of an element at a given location in a saline aquifer of a respective basin. The machine-learning method comprises, for each basin and with respect to a predetermined set of one or more geochemical variables, providing a dataset, and learning the predictive basin-wise model based on the dataset. The dataset comprises, for respective saline aquifer locations of the basin, training samples. Each training sample includes a measurement of one or more geochemical variables of the predetermined set. Each training sample further includes a respective ground truth value. The ground truth value represents a concentration of the element at the respective saline aquifer location.

In examples, the method machine-learning may comprise any one or more of the following:
the element is a metal, for example lithium;
the predetermined set of one or more geochemical variables comprises a concentration of any one or any combination of the following chemical elements: Cl, Ca, Na, B, Mg, Sr, and/or K;
each predictive basin-wise model comprises an ensemble-learning model, for example a tree-based model, such as an XG boost model or a Random Forest model;
each predictive basin-wise model comprises respective alternative sub-models, each sub-model being configured for predicting the concentration of the element at the given location in the saline aquifer when inputted with a measurement of a respective combination of the one or more geochemical variables, each sub-model being learnt on corresponding portions of the training samples of the dataset; and/or
the dataset comprises missing values, and the learning of each respective sub-model is based on training samples having no missing value in the portion thereof corresponding to the respective sub-model.

It is further provided a computer-implemented method of using a predictive basin-wise model learnt (i.e. having been learnt) according to the machine-learning method, referred hereinafter as to the “prediction method”. The prediction method is for predicting the concentration of the element at the given location in the saline aquifer of the basin respective to the predictive basin-wise model. The prediction method comprises providing a given measurement of one or more geochemical variables of the predetermined set at the given location, and predicting the concentration of the element at the given location by applying the predictive basin-wise model to the given measurement.

In examples, the predictive basin-wise model is learnt (e.g. has been learnt) according to any example of the machine-learning method wherein the dataset comprises missing values and the learning of each respective sub-model is based on training samples having no missing value in the portion thereof corresponding to the respective sub-model. In such a case, the one or more geochemical variables of the given measurement in the prediction method form a portion of the predetermined set, and the predicting of the concentration of the element at the given location may optionally comprise applying the sub-model of the predictive basin-wise model corresponding to said portion of the predetermined set.

In examples, the prediction method further comprises providing a plurality of predictive basin-wise models each learnt according to the machine-learning method, providing a value of one or more geographical variables representing a given location, based on the value of the one or more geographical variables, determining a given basin corresponding to the given location, selecting the predictive basin-wise model corresponding to the given basin, and predicting the concentration of the element at the given location by applying the selected predictive basin-wise model to the provided measurement of the one or more geochemical variables.

It is further provided a data structure (i.e. recorded or recordable data specifying/representing information) comprising a plurality of predictive basin-wise models learnt (i.e. having been learnt) by performing the machine-learning method. Additionally or alternatively, the data structure may comprise a computer program comprising instructions for performing the machine-learning method and/or the prediction method.

It is further provided a device comprising a computer readable storage medium having recorded thereon the data structure. The device may form or serve as a non-transitory computer-readable medium, for example on a Saas (Software as a service) or other server, or a cloud based platform, or the like. The device may alternatively comprise a processor coupled to the data storage medium. The device may thus form a computer system in whole or in part (e.g. the device is a subsystem of the overall system). The system may further comprise a graphical user interface coupled to the processor.

It is further provided a process for storing CO2 in a saline aquifer of a respective basin process. The process comprises predicting a concentration of an element in one or more first locations of the saline aquifer using the prediction method. The process also comprises determining one or more second locations of the saline aquifer for storing CO2 based on the prediction. The process may further comprise storing CO2 in least one second location. The storing includes withdrawing water from the at least one second location. The process may further comprise recovering the element in the withdrawn water.

### BRIEF DESCRIPTION OF THE DRAWINGS

Non-limiting examples will now be described in reference to the accompanying drawings, where:

FIG. 1 shows an example of the system; and

FIGS. 2 to 6 illustrate the methods.

### DETAILED DESCRIPTION

It is hereby proposed a computer-implemented method of machine-learning a plurality of predictive basin-wise models. Each predictive basin-wise model is configured for predicting a concentration of an element at a given location in a saline aquifer of a respective basin. The machine-learning method comprises, for each basin and with respect to a predetermined set of one or more geochemical variables, providing a dataset, and learning the predictive basin-wise model based on the dataset. The dataset comprises, for respective saline aquifer locations of the basin, training samples. Each training sample includes a measurement of one or more geochemical variables of the predetermined set. Each training sample further includes a respective ground truth value. The ground truth value represents a concentration of the element at the respective saline aquifer location.

Such a method forms an improved solution for analysis of a saline aquifer with respect to a given element of interest.

The method indeed allows learning/training a plurality of predictive basin-wise models. Each predictive basin-wise model may then be used in a computer-implemented method for predicting the concentration of the element at the given location in the saline aquifer of the basin respective to which the predictive basin-wise model has been learnt. The prediction method comprises providing a given measurement of one or more geochemical variables of the predetermined set at the given location, and predicting the concentration of the element at the given location by applying the predictive basin-wise model to the given measurement. The prediction method may comprise the method of machine-learning, or it may be performed separately afterwards.

Such a prediction method allows predicting the concentration of the element at said given location without having to directly measure it. Direct measurement would for example comprise using Inductively coupled plasma (ICP) mass spectrometry techniques. This is can be costly, in particular when the element is a metal. This can also be cumbersome, in particular in applications where it is desired to estimate the concentration of the element several (e.g. many) times, for example at distinct locations of a saline aquifer. In such a case, a more effective process may comprise performing several times the prediction method, for example each time at a respective one of the distinct locations. Thanks to the prediction being performed by applying the predictive basin-wise model to the given measurement of one or more geochemical variables, the predicted concentration is relatively accurate and outputted relatively fast. It has indeed been found that local values of geochemical variables can be accurate predictors of the local concentration of the element at a given location in a saline aquifer. Further, the given measurement of one or more geochemical variables may be relatively fast to perform and/or to retrieve from past measurements. e.g. stored in one or more databases.

In addition, the proposed solution does not require any predetermination of the relationship (e.g. correlation) between the one or more geochemical variables and the concentration of the element, as said relationship is automatically determined by machine-learning. Now, it has been identified that such a relationship depends on the respective (geological) basin of each saline aquifer. In fact, it has been found that correlation between the one or more geochemical variables and the concentration of the element may be relatively low on a worldwide scale, but may surprisingly become relatively high on the scale of basins. Thus, by learning a plurality of predictive models with basin-wise training schemes and based on basin-wise datasets, the proposed solution allows a geographical specialization of the machine-learning on the scale of geological basins. Relative to a machine-learning scheme that would be performed on a worldwide scale, such a geographical specialization allows the model to actually learn to predict the concentration of the element from geochemical measurements, or at least to learn to perform such a prediction more accurately. In a sense, the proposed solution puts the learning focus each time on relevant parts of the data (i.e. each individual basin-wise dataset) such that the learning performs better. Yet, the proposed solution stays at a large enough scale, that is, at the scale of basins (rather than individual saline aquifers for example), such that it is easier for the datasets to be large enough and to present sufficient diversity to achieve a robust learning. Where the low global correlation would dissuade from applying a machine-learning solution at all, the retained basin-wise specialization actually allows for achieving relatively high accuracy in the eventual predictions.

In an example, the prediction method is performed by a computer system and comprises providing, on the computer system or on a remote server connected to the system, the plurality of predictive basin-wise models that have been learnt according to the machine-learning method. The prediction method further comprises providing on the system a value of one or more geographical variables representing a given location. The prediction method further comprises, based on the value of the one or more geographical variables, determining a given basin corresponding to the given location. The value of one or more geographical variables indeed forms data indicative of the basin to which belongs the given location at which the provided given measurement (provided in the prediction method) was performed. The value of one or more geographical variables may for example comprise geographical coordinates (e.g. including latitude, longitude, and/or depth), tags (e.g. basin name from a predetermined nomenclature), and/or numeric identifiers (e.g. indices). The prediction method may for example comprise comparing the value of the one or more geographical variables to a database (stored locally on the system or remotely on a server) to determine the basin in which the concerned aquifer is located. The predicting of the concentration of the element at the given location may then comprise determining the corresponding predictive basin-wise model (from the provided plurality of predictive basin-wise models) before applying said selected model to the given measurement. In other words, the prediction method comprises selecting and applying, among the plurality of models, the predictive basin-wise model that was learnt for the determined basin (i.e. based on the dataset with the saline aquifer locations of the basin).

The prediction method may be used in different applications, including improved recovery of the element in the saline aquifer, for example in the context of a carbon dioxide (CO2) storage process.

Such a process may comprise predicting a concentration of an element in one or more first locations of a saline aquifer using the prediction method. The process also comprises determining, based on the prediction, one or more second locations (of the saline aquifer) for storing CO2. The one or more first locations may be referred to as “testing locations” and are locations of the saline aquifer each at which a given measurement of one or more geochemical variables of the predetermined set may be provided so as to perform the prediction method. The one or more second locations may be referred to as “locations optimal for CO2 storage” and may comprise one or more of the first locations, and/or one or more distinct locations determined from the one or more of the first locations, for example by interpolation or barycenter calculation. The one or more second locations may be locations that are optimal for the CO2 storage based on the predicted concentration of the element thereat.

For example, the element may have a certain value and the process may comprise producing a quantity of the element (i.e. outputting the element in a usable form, e.g. after having purified the element). In such a case, the one or more second locations may be first locations which maximize the predicted concentration of the element (i.e. the result of the prediction method is maximal at said first locations). In other examples, other factors may be taken into account, for example difficulty in storing CO2 at a given location and/or quantity of storage space available. In such a case, the determining of the one or more second locations may comprise optimizing an objective function which not only rewards (i.e. positively takes into account) the predicted concentration of the element, but also rewards or penalizes other factors.

In an another example, the element may be hazardous and be sought to be recovered merely to depollute the withdrawn water. The prediction method may in such a case be integrated into a CO2 storage feasibility study for a recovery process of the element from the withdrawn water. The feasibility study may decide, based on the result of the prediction, if the concentration of the element in the produced water of the production is below a necessary threshold to be feasibly or easily extractable. The one or more second locations may be first locations which minimize the predicted concentration of the element in such a case.

Such a process thus allows storing CO2 at locations of the saline aquifer that are optimal with respect to the predicted concentration of the element, so as to potentially recover or have to recover an optimal quantity of the element upon the CO2 storage. The process may indeed further comprise storing CO2 in at least one second location. This means that CO2 is injected (e.g. from a well. e.g. having been drilled therefore) at said at least one second location so as to be kept/imprisoned there. The storing includes withdrawing water from the at least one second location. This means that water of the saline aquifer contained at said at least one second location is expelled due to the injected CO2 and the water is further received to be processed, for example at a water treatment installation. e.g. on ground surface. The process may further comprise recovering (i.e. extracting/collecting) the element in the withdrawn water. The recovery process may be any adapted process depending on the type and/or quantity of the element. Thanks to the prediction method, the process may optimize the quantity of the element recovered for a given quantity of CO2 to be stored.

A basin, also referred to as “geological basin” or “sedimentary basin”, is a large area presenting a geological coherence and comprising one or more saline aquifers. Any basin herein may be any basin of any known nomenclature of basins from geoscience literature. The plurality of basins (corresponding to the plurality of predictive basin-wise models) may for example comprise or consist of any subset from the basins defined in the Robertson classification. The plurality of basins may consist of distinct basins, at least two of which (e.g. all) being geographically non-overlapping or substantially non-overlapping. At least one (e.g. each) of the basins may comprise several distinct saline aquifers.

The proposed solution allows to determine local concentrations of the element at different positions of saline aquifers. Thus, at least one (e.g. each) dataset in the machine-learning method may comprise, for at least one (e.g. each) saline aquifer of the respective basin, at least two different training samples (i.e. corresponding to at least two distinct saline aquifer locations. i.e. different positions in the saline aquifer) with different values for the measurement of one or more geochemical variables (e.g. including different values of at least one same geochemical variable), and/or different ground truth values. Similarly, the prediction method may be performed at least at two distinct locations of a same saline aquifer, based on different values of the given measurement and/or resulting in different predicted concentrations. As a result the process for storing CO2 may involve a plurality of distinct first locations of a same saline aquifer, with different prediction results, the process thus selecting an optimal location.

The element of which concentration is learnt to be predicted may be any element of interest known to be potentially contained in saline aquifers. The element may for example be a metal, such as lithium. It has been found and experimentally observed that the concentration of lithium in saline aquifers may be accurately predicted by a measurement of one or more geochemical variables, as shown later. The element may alternatively be one of cobalt, nickel, or cadmium. Such examples of the element are known to involve complex techniques for measuring their concentration in a sample of water, such as the earlier-mentioned ICP mass spectrometry technique. While such techniques may be involved or have been involved offline in the construction of the datasets used in the machine-learning method, it is inefficient to implement them in an online prediction scenario where a more real-time response is expected. “Offline” refers to a time prior to or during the machine-learning phase, and “online” refers to a time during the prediction phase.

The proposed solution thus rather builds upon a predetermined set of one or more geochemical variables in order to perform a prediction of the element's concentration. A geochemical variable is a variable that represents a local parameter chemically measurable at a given location of the subsoil, such as at a given location in a saline aquifer. By “chemically measurable”, it is meant that the local value of the parameter can be determined essentially via a chemical reaction, such that the measuring is relatively simple to perform. This thus excludes mass spectrometry techniques such as ICP mass spectrometry.

The prediction method may comprise performing such a chemical reaction for at least one (e.g. each) provided geochemical variable to obtain the given measurement. Alternatively, the given measurement may have been obtained beforehand in that manner, and the prediction method may comprise retrieving the value. e.g. from local or distant memory. Similarly, the machine-learning method may comprise performing such a chemical reaction for geochemical variable measurements in the dataset, and/or retrieving geochemical variable measurements having been obtained beforehand in that manner. Yet alternatively, the machine-learning method may comprise retrieving a dataset having been populated in such manners.

In an example, the predetermined set of one or more geochemical variables may comprise or consist of a concentration of one or more chemical elements (all distinct from the element of which concentration is learnt to be predicted). At least one (e.g. each) such concentration may be measurable by a respective precipitation reaction. The measurement may in such a case comprise obtaining an initial solution of a known volume containing a given chemical element in a solute form, precipitating the chemical element by adding to the solution any known reactant of the chemical element, and measuring the quantity of the reactant at which precipitation stops. This allows for the determination of the concentration of the given chemical element in the initial solution based on said measured quantity and on the initial volume. Such a precipitation measurement is relatively simple and fast to perform. Additionally or alternatively, at least one (e.g. each) such concentration may be measurable by a respective chromatography method (e.g. ion chromatography). Such a chromatography measurement is relatively simple and fast to perform.

The prediction method may comprise performing such a precipitation measurement, and/or a chromatography measurement for at least one (e.g. each) provided geochemical variable to obtain the given measurement, or retrieving a given measurement having been obtained in that manner. Similarly, any geochemical variable measurement in the dataset may be obtained, during the machine-learning method, or may have been obtained, prior to the machine-learning method, by way of such a precipitation measurement and/or a chromatography measurement. Yet alternatively, the machine-learning method may comprise retrieving a dataset having been populated in such manners.

The prediction method may comprise sampling water (i.e. brine) at the given location of the saline aquifer by any sampling technique, or providing water having been sampled by such sampling technique. Yet alternatively, in options where the prediction method comprises retrieving the given measurement. e.g. from local or distant memory, the given measurement may have been performed on water having been sampled by such sampling technique. The sampling may comprise drilling a well arriving at or passing by the given location, or using such an existing well, so as to enable extraction of water from the given location. The prediction method may comprise moving the sampled water to the surface and performing the (e.g. precipitation) measurement(s) thereon. Similarly, the machine-learning method may comprise such a sampling scheme to populate the dataset, or retrieving data having been obtained from such a sampling scheme to populate the dataset. Yet alternatively, the machine-learning method may comprise retrieving a dataset having been populated in such manners.

The predetermined set of one or more geochemical variables may comprise a concentration of any one or any combination (e.g. all) of the following chemical elements: Cl. Ca. Na. B. Mg. Sr. and/or K. It has been found that knowledge of the local concentration of different combinations of these chemical elements in a saline aquifer allows to accurately predict the concentration of the element of interest, in particular when the element is a metal, and more particularly when the element is lithium.

Thanks to the use of a machine-learning framework, each predictive basin-wise model may be configured automatically via the learning (also referred to as “training”). In other words, all parameters of the predictive basin-wise model need not be predetermined by a user, as at least part of them are adjusted automatically during the learning process via a training of an (e.g. initially untrained) model. In addition, at least one (e.g. each) predictive basin-wise model may comprise a non-linear function that relates (non-linearly) the input (i.e. the given measurement of one or more geochemical variables) to the output (i.e. the concentration of the element), such that complex input-output relationships can be captured.

The learning of each predictive basin-wise model is performed thanks to the presence of a dataset that contains so-called “training samples”, also referred to as “examples”, that are merely pieces of data that each relate a respective value of the input to a corresponding value of the output known to be true for that input value, the so-called “ground truth value”. The learning may comprise adjusting parameter values of the predictive basin-wise model such that the prediction performs well on the dataset. In other words, the adjustment is such that, once performed, applying the predictive basin-wise model to the measurements contained in the dataset yields a prediction as accurate as possible, that is, as close as possible to the corresponding ground truth values contained in the dataset. The learning may for example comprise providing a model architecture having free parameters (i.e. adjustable parameters), and then training the model based on the dataset. The training may comprise one or more minimization schemes, each including varying the free parameters until they minimize a predetermined prediction loss evaluated based on the dataset. As known, the dataset may be divided into a training dataset and a testing dataset.

The dataset contains diverse training samples so as to represent the diversity of the situations with different input-output relations, such that the learning may be efficient, that is, result in an accurate predictor. Each basin-wise dataset may for example comprise at least 50 training samples. To populate the dataset, the machine-learning method may comprise determining at least part of the ground truth values by a measurement on the sampled water according to any known method adapted to estimate the concentration of the element (e.g. metal), for example a mass spectrometry technique such as ICP mass spectrometry. Alternatively or additionally, the machine-learning method may comprise retrieving ground truth values having been obtained in that manner, to populate the dataset. Yet alternatively, the machine-learning method may comprise retrieving a dataset having been populated in such manners.

The machine-learning method is for learning a plurality of predictive basin-wise models, each corresponding to a respective distinct basin of a plurality of basins. The datasets provided in the machine-learning method pertain to different and geographically distinct basins. Each training sample indeed comprises data related to a respective saline aquifer location, thus belonging to one and only one of the plurality of basins. The measurement of the one or more geochemical variables is indeed a measurement of the value of said variables at the respective location, and the ground truth value similarly represents a concentration of the element at the respective location, thus both in a respective basin.

The machine-learning method then performs independently the learning of the different predictive basin-wise models each based on its respective dataset, for example in parallel or sequentially. A consequence of such “independent” learning is that, for any given predictive basin-wise model and its respective dataset, modifying values in other datasets would have no impact on the result. In other words, as long as the respective dataset would be left unmodified, the given predictive basin-wise model would be the same after the learning/training whatever the modifications elsewhere may be (whichever changes to the other datasets). Yet in other words, changing values of training samples of a given dataset has no impact to predictive basin-wise models other than the model respective to the given dataset. For example, the learning/training of each predictive basin-wise model may comprise one or more respective minimizations of a loss defined based on a respective dataset, and the minimizations pertaining to different basins may be performed independently one from another. The predictive basin-wise models may all present the same model architecture and/or the losses may all consist of the same function, but the parameters of the predictive basin-wise models may have different values, after all the learnings/trainings, since the adjustments of the parameter values are different, due to the datasets they are based upon being different.

Each predictive basin-wise model may comprise at least one ensemble-learning model. It has been found that ensemble learning applies particularly well to the problem at stake. Each ensemble-learning model may in specific be a tree-based model. e.g. having different trees that vote to decide among them which one outputs the best prediction. Each tree-based model may for example be an XG boost model or a Random Forest model. These two types of decision tree models have been found to achieve particularly high accuracy in the predictions. It is believed that this is due to the predictability of the concentration of the element (in particular when the element is a metal, such as lithium) being related to local chemistry at a location of the saline aquifer, and that such local chemistry presents little spatial consistency that deep-learning techniques such as convolutional neural networks (CNNs) would typically capture. As a result, ensemble-learning models and in particular tree-based models such as XG boost or a Random Forest achieve higher accuracy. These models further allow the datasets to be relatively sparsely populated.

The predetermined set may comprise a plurality of geochemical variables, for example a concentration of each of a plurality of chemical elements, such as comprising or consisting of any combination of several (e.g. all) of the following elements: Cl. Ca. Na. B. Mg. Sr. and/or K.

The prediction method may comprise providing a given measurement of each geochemical variable of the predetermined set at the given location. Thus, the predictive basin-wise model is applied to a maximal quantity of information. Similarly, at least a portion (e.g. all) of the training samples of each dataset may comprise, per training sample, a measurement of each geochemical variable of the predetermined set.

Alternatively, the prediction method may comprise providing a given measurement of a portion (i.e. a subpart) of the geochemical variables of the predetermined set at the given location. In other words, for at least one geochemical variable of the predetermined set, the measurement value is missing. This may for example occur in different situations, for example when the prediction method is performed by retrieving measurements performed in past studies of saline aquifers, but not all measurements were performed and none of the sampled water is available anymore. This may also occur when the dataset used for a certain basin of the plurality of basins comprises only few or no measurements for one or more of the predetermined set of geochemical variables. In such a situation, only little may be learnt for such unrepresented or low-represented variables, such that they possess to or too little prediction power. As a result, the prediction method may ignore the measurement provided for such variables (if any) even if available, when it is desired to use the prediction method for that same basin.

In all such situations, the proposed solution may comprise a countermeasure that allows the method to still perform a relatively accurate prediction. Namely, at least one (e.g. each) predictive basin-wise model may comprise a plurality of respective alternative sub-models. Each sub-model is configured for being used alternatively (i.e. selectively) to predict the concentration of the element at the given location in the saline aquifer when inputted with a measurement of a respective combination of the one or more geochemical variables. The prediction method may thereby comprise predicting the concentration of the element at the given location by applying the sub-model that corresponds to said portion of the predetermined set for which a measurement is available.

Such sub-models may be learnt/trained using corresponding portions of the training samples of the dataset. The machine-learning method may comprise learning the sub-models independently one from another, each time retaining only the relevant portion of data provided by each training sample of the dataset. For each sub-model configured to be applied to a measurement of a respective combination of the geochemical variables, the learning/training of the sub-model may merely comprise discarding/ignoring values of each training sample representing a measurement of another geometrical variable (i.e. not part of the respective combination). The sub-models are thus of the same nature and are essentially predictive basin-wise models, and they differ by being applicable to different inputs.

The sub-models may each present the same model architecture. Each sub-model may be an ensemble-learning model. Each ensemble-learning model may be a tree-based model, for example an XG boost model or a Random Forest model. The sub-models may all be XG boost models, or all Random Forest models, or vet some sub-models may be XG boost models while other sub-models may be Random Forest models.

An example is discussed where the predetermined set comprises a concentration of each the seven following elements: Cl. Ca. Na. B. Mg. Sr. and K. In such an example, each predictive basin-wise model may comprise a number of sub-models equal to 2⁷-1, each corresponding to a respective combination of one or more of the seven elements. The machine-learning may comprise learning each sub-model independently. For example, during the learning of the sub-model corresponding to the combination of the concentration of Cl. Ca. Na. and B, each training sample having a value for these four elements remains a “valid” training sample (i.e. adapted for machine-learning), since the concentrations of Mg. Sr. and K (if any) may simply be ignored.

Still in all such situations, at least a portion (e.g. all) of the training samples of each dataset may optionally comprise, per training sample, a measurement of each geochemical variable of the predetermined set.

Alternatively or additionally, at least one (e.g. each) dataset provided in the machine-learning method may comprise missing values. In other words, for each of a portion of the training samples of the dataset, the training sample is provided with no value for at least one respective geometrical variable. The missing geometrical variables may be different from one training sample to another, and further from one dataset to another. In such a case, the machine-learning method may comprise learning each respective sub-model based on training samples having no missing value in the portion thereof corresponding to the respective sub-model. In other words, for each respective sub-model, only those training samples of the dataset that initially have a measurement value for each geometrical variable involved in the respective sub-model are used in the training. Relative to an alternative scheme which would comprise filling the missing value, such learning by rather restricting the dataset allows to achieve higher accuracy. Measurements of geochemical variables at locations of saline aquifers are relatively scarce data, and the level of scarcity is actually such that filling missing values would introduce biases that would be too high in the later predictions. It has been found that higher accuracy can be achieved by rather proceeding with sub-models and restricting the learning to reliable data.

Each method herein is computer-implemented. This means that steps (or substantially all the steps) of the method are executed by at least one computer, or any system alike. Thus, steps of the method are performed by the computer, possibly fully automatically or semi-automatically. In examples, the triggering of at least some of the steps of the method may be performed through user-computer interaction. The level of user-computer interaction required may depend on the level of automatism foreseen and put in balance with the need to implement user's wishes. In examples, this level may be user-defined and/or pre-defined.

A typical example of computer-implementation of a method is to perform the method with a system adapted for this purpose. The system may comprise a processor coupled to a memory and a graphical user interface (GUI), the memory having recorded thereon a computer program comprising instructions for performing the method. The memory may also store a database. The memory is any hardware adapted for such storage, possibly comprising several physical distinct parts (e.g. one for the program, and possibly one for the database).

By “database”, it is meant any collection of data (i.e. information) organized for search and retrieval (e.g. a relational database. e.g. based on a predetermined structured language. e.g. SQL). When stored on a memory, the database allows a rapid search and retrieval by a computer. Databases are indeed structured to facilitate storage, retrieval, modification, and deletion of data in conjunction with various data-processing operations. The database may consist of a file or set of files that can be broken down into records, each of which consists of one or more fields. Fields are the basic units of data storage. Users may retrieve data primarily through queries. Using keywords and sorting commands, users can rapidly search, rearrange, group, and select the field in many records to retrieve or create reports on particular aggregates of data according to the rules of the database management system being used.

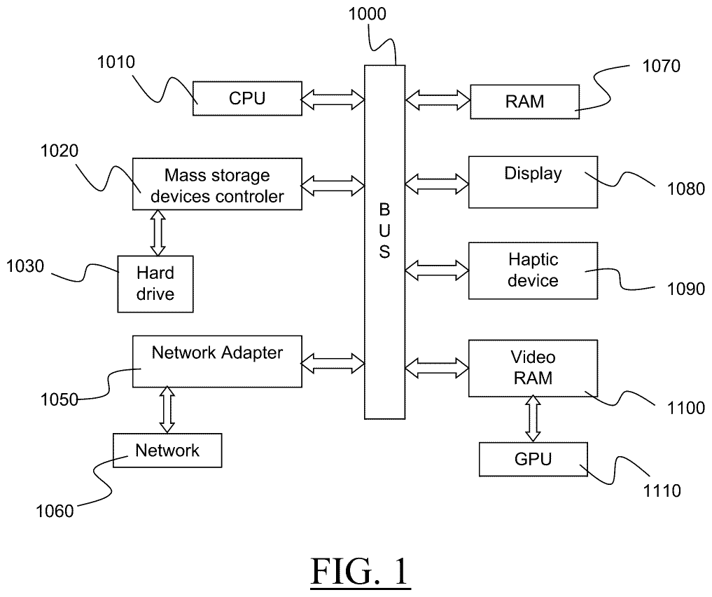

FIG. 1 shows an example of the system, wherein the system is a client computer system. e.g. a workstation of a user.

The client computer of the example comprises a central processing unit (CPU) 1010 connected to an internal communication BUS 1000, a random-access memory (RAM) 1070 also connected to the BUS. The client computer is further provided with a graphical processing unit (GPU) 1110 which is associated with a video random access memory 1100 connected to the BUS. Video RAM 1100 is also known in the art as frame buffer. A mass storage device controller 1020 manages accesses to a mass memory device, such as hard drive 1030. Mass memory devices suitable for tangibly embodying computer program instructions and data include all forms of nonvolatile memory, including by way of example semiconductor memory devices, such as EPROM. EEPROM, and flash memory devices: magnetic disks such as internal hard disks and removable disks: magneto-optical disks; and CD-ROM disks 1040. Any of the foregoing may be supplemented by, or incorporated in, specially designed ASICs (application-specific integrated circuits). A network adapter 1050 manages accesses to a network 1060. The client computer may also include a haptic device 1090 such as cursor control device, a keyboard or the like. A cursor control device is used in the client computer to permit the user to selectively position a cursor at any desired location on display 1080. In addition, the cursor control device allows the user to select various commands, and input control signals. The cursor control device includes a number of signal generation devices for input control signals to system. Typically, a cursor control device may be a mouse, the button of the mouse being used to generate the signals. Alternatively or additionally, the client computer system may comprise a sensitive pad, and/or a sensitive screen.

The computer program may comprise instructions executable by a computer, the instructions comprising means for causing the above system to perform the method. The program may be recordable on any data storage medium, including the memory of the system. The program may for example be implemented in digital electronic circuitry, or in computer hardware, firmware, software, or in combinations of them. The program may be implemented as an apparatus, for example a product tangibly embodied in a machine-readable storage device for execution by a programmable processor. Method steps may be performed by a programmable processor executing a program of instructions to perform functions of the method by operating on input data and generating output. The processor may thus be programmable and coupled to receive data and instructions from, and to transmit data and instructions to, a data storage system, at least one input device, and at least one output device. The application program may be implemented in a high-level procedural or object-oriented programming language, or in assembly or machine language if desired. In any case, the language may be a compiled or interpreted language. The program may be a full installation program or an update program. Application of the program on the system results in any case in instructions for performing the method. The computer program may alternatively be stored and executed on a server of a cloud computing environment, the server being in communication across a network with one or more clients. In such a case a processing unit executes the instructions comprised by the program, thereby causing the method to be performed on the cloud computing environment.

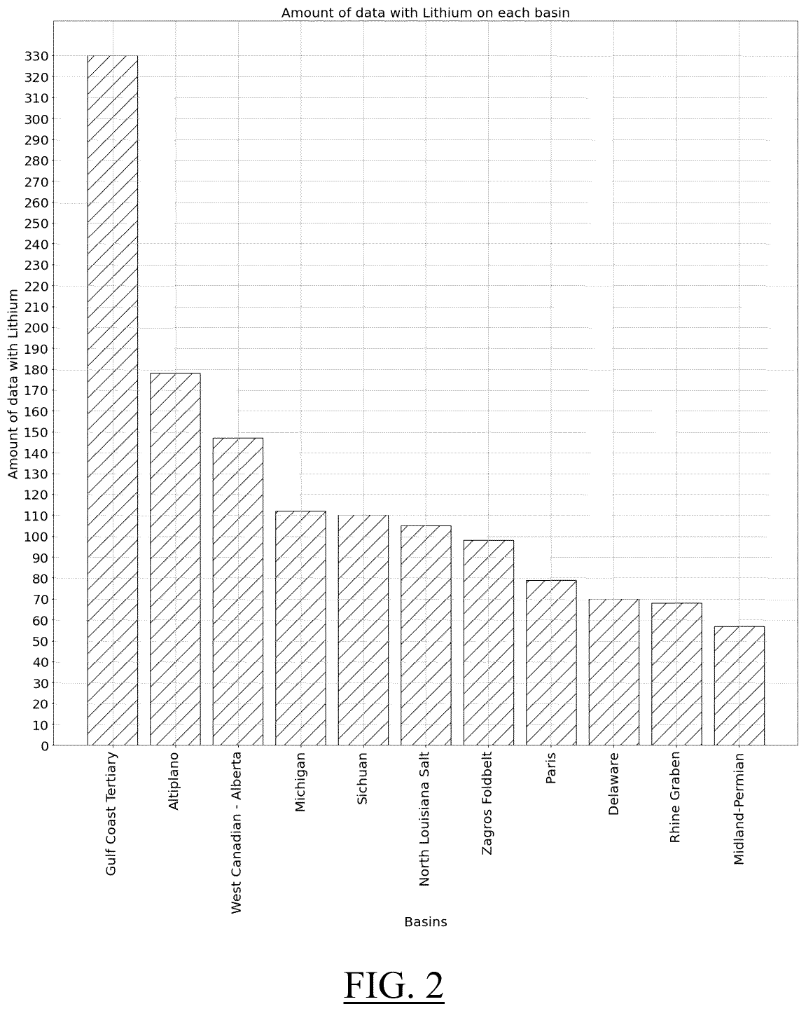

FIGS. 2-6 illustrate accuracy of the prediction allowed by the method, based on real data that were obtained and on calculations and/or tests that were performed. In FIGS. 2 to 6, the element concentration of interest is that of Lithium (mg/L). The dataset comprises global values resulting from the geochemical analysis of eleven basins.

FIG. 2 shows the amount of data that was obtained for each of the eleven basins. The diagram shows in particular per basin the count of training samples that were obtained containing each a concentration of at least a combination of the seven following chemical elements: Cl, Ca, Na, B, Mg. Sr, and/or K, and each a ground truth value representing a concentration of Li, each training sample corresponding to a respective saline aquifer location of the basin. These training samples were obtained by retrieving data from public results of past prospections of saline aquifers, including results as described in paper Dugamin, E. J. M., Richard, A., Cathelineau, M. et al. Groundwater in sedimentary basins as potential lithium resource: a global prospective study. Sci Rep 11, 21091 (2021). https://doi.org/10.1038/s41598-021-99912-7 and/or results presented in other papers referred to in that paper.

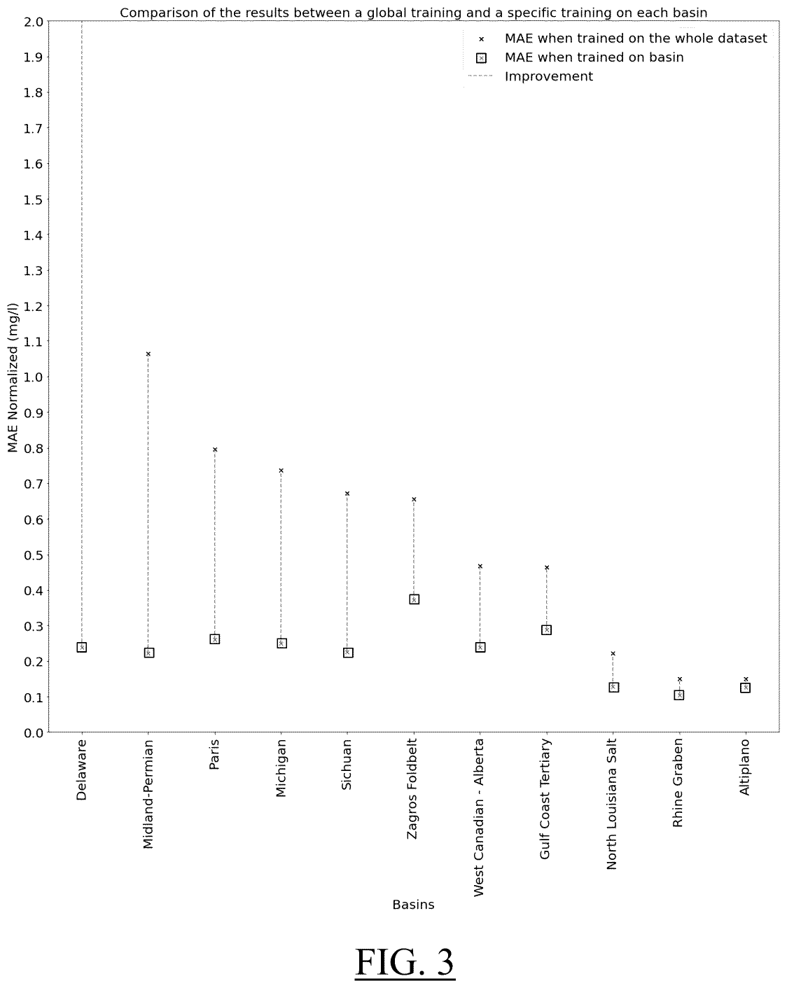

FIG. 3 shows results for an implementation of the methods as previously described, where an XG Boost architecture was used for training each sub-model. The method comprised model use of training samples relating respective input values to a respective ground truth value of Lithium concentration (mg/L). The normalized mean absolute error (MAE) was the error type used to train and validate the models. MAE is the absolute distance between the actual (ground truth) value of the concentration of the element and the prediction. Comparisons were made between training on a global worldwide dataset and training on specific basin-wise datasets. The MAE for the output values per basin was calculated for both approaches under the same conditions. Improvement lines are mapped between plotted results. Training executed on specific datasets for each basin is shown to provide better results for all basins, and considerably better results for most basins. The implementation results of FIG. 3 are further presented in Table 1 below:

TABLE 1

| | MAE when | MAE when | | Mean Li | | |
|---|---|---|---|---|---|---|
| | Trained on | trained on | | (mg/l) | | |
| | a specific | the global | Number | on each | MAE_basin/ | MAE_global/ |
| Basin | basin | dataset | of data | basin | Mean Li | Mean Li |
| Rhine Graben | 11.036 | 15.885 | 61 | 106.04 | 0.104 | 0.15 |
| Altiplano | 86.31 | 103.338 | 178 | 689.48 | 0.125 | 0.15 |
| North | 21.158 | 36.694 | 83 | 164.99 | 0.128 | 0.222 |
| Louisiana Salt | | | | | | |
| Midland- | 1.093 | 5.264 | 51 | 4.95 | 0.221 | 1.063 |
| Permian | | | | | | |
| Sichuan | 5.801 | 17.344 | 109 | 25.78 | 0.225 | 0.673 |
| Delaware | 1.065 | 116.543 | 62 | 4.46 | 0.239 | 26.131 |
| West | 7.832 | 15.317 | 144 | 32.77 | 0.239 | 0.467 |
| Canadian - | | | | | | |
| Alberta | | | | | | |
| Michigan | 10.273 | 30.341 | 112 | 41.22 | 0.249 | 0.736 |
| Paris | 2.033 | 6.196 | 77 | 7.79 | 0.261 | 0.795 |
| Gulf Coast | 6.254 | 10.122 | 311 | 21.79 | 0.287 | 0.465 |
| Tertiary | | | | | | |
| Zagros | 3.312 | 5.835 | 94 | 8.9 | 0.372 | 0.656 |
| Foldbelt | | | | | | |

Similarly, Tables 2 and 3 below show correlations that were calculated based on the obtained data. These tables explain why a much higher correlation with Lithium may be achieved by applying a model to each basin as opposed to a global dataset. Table 2 displays the best correlation with Lithium for datasets pertaining to each basin (among the correlations computed for each of the seven chemical elements). Table 3 displays the correlations with Lithium for a worldwide dataset.

TABLE 2

| | Best Correlation |
|---|---|
| Basins | with Lithium |
| Sichuan | 0.972 |
| North Louisiana Salt | 0.969 |
| Altiplano | 0.958 |
| Midland-Permian | 0.937 |
| Zagros Foldbelt | 0.927 |
| Rhine Graben | 0.906 |
| Gulf Coast Tertiary | 0.861 |
| West Canadian - Alberta | 0.847 |
| Delaware | 0.839 |
| Michigan | 0.816 |
| Paris | 0.812 |

TABLE 3

| | Correlation with |
|---|---|
| Variables | Li (mg/l) - Worldwide |
| B (mg/l) | 0.688 |
| Sr (mg/l) | 0.506 |
| Cl (mg/l) | 0.430 |
| K (mg/l) | 0.382 |
| Na (mg/l) | 0.354 |
| Mg (mg/l) | 0.322 |
| Ca (mg/l) | 0.039 |

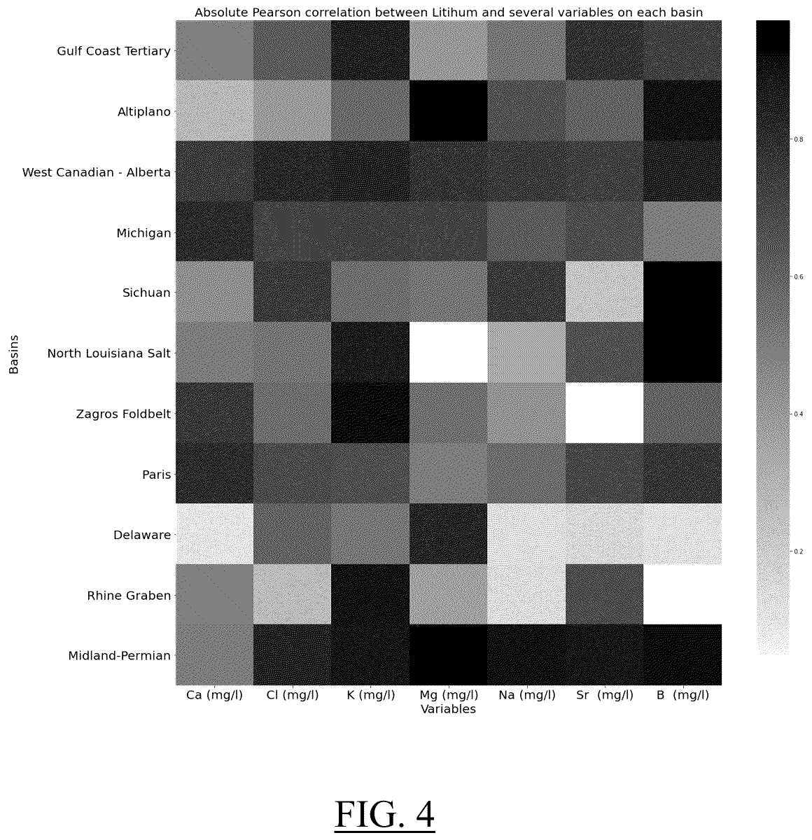

Values of Table 2 may be observed graphically in FIG. 4, demonstrating an absolute Pearson correlation between Lithium and several variables in each basin. For example, a correlation value of 0.861 was obtained for K for the Gulf Coast Tertiary (Table 2, FIG. 4), whereas a value of only 0.354 was obtained worldwide (Table 3).

Meanwhile, Table 4 below provides values for mean and median correlation values between Lithium and each element Cl, Ca, Na, B, Mg, Sr and K across all basins and for which separate models were applied. Correlations are clearly improved for the basin-wise values of Table 4 versus those displayed in the worldwide dataset of Table 3.

TABLE 4

| | Mean correlation with | Median correlation with |
|---|---|---|
| Variables | Lithium Over basins | Lithium Over basins |
| K (mg/l) | 0.704 | 0.761 |
| B (mg/l) | 0.619 | 0.720 |
| Sr (mg/l) | 0.614 | 0.706 |
| Cl (mg/l) | 0.564 | 0.570 |
| Ca (mg/l) | 0.553 | 0.505 |
| Na (mg/l) | 0.517 | 0.504 |
| Mg (mg/l) | 0.512 | 0.464 |

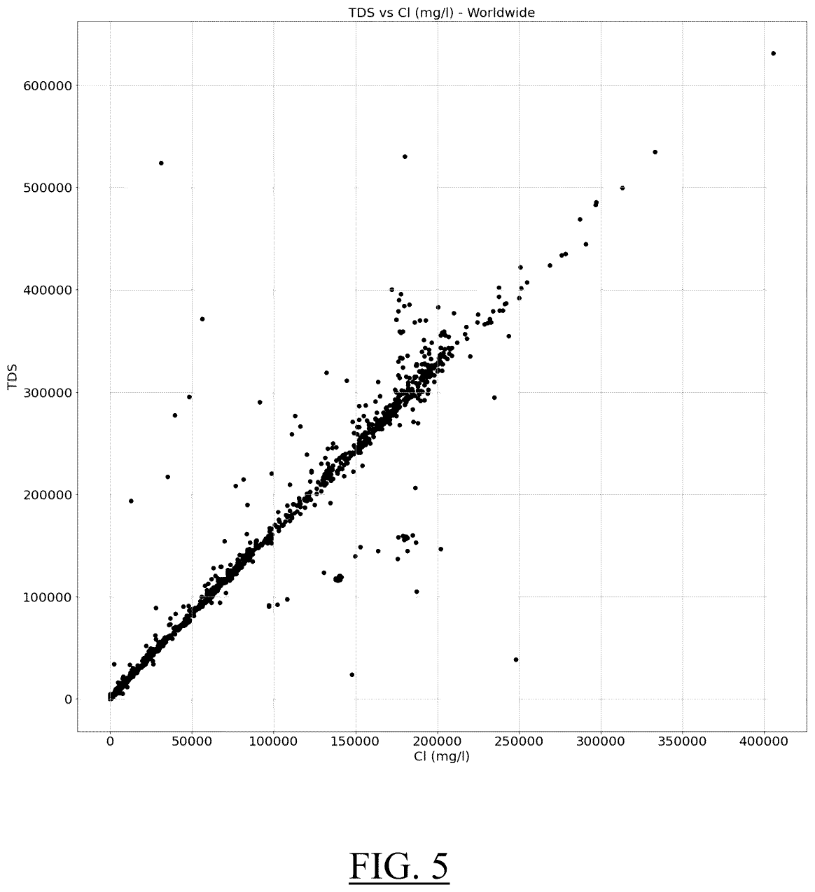

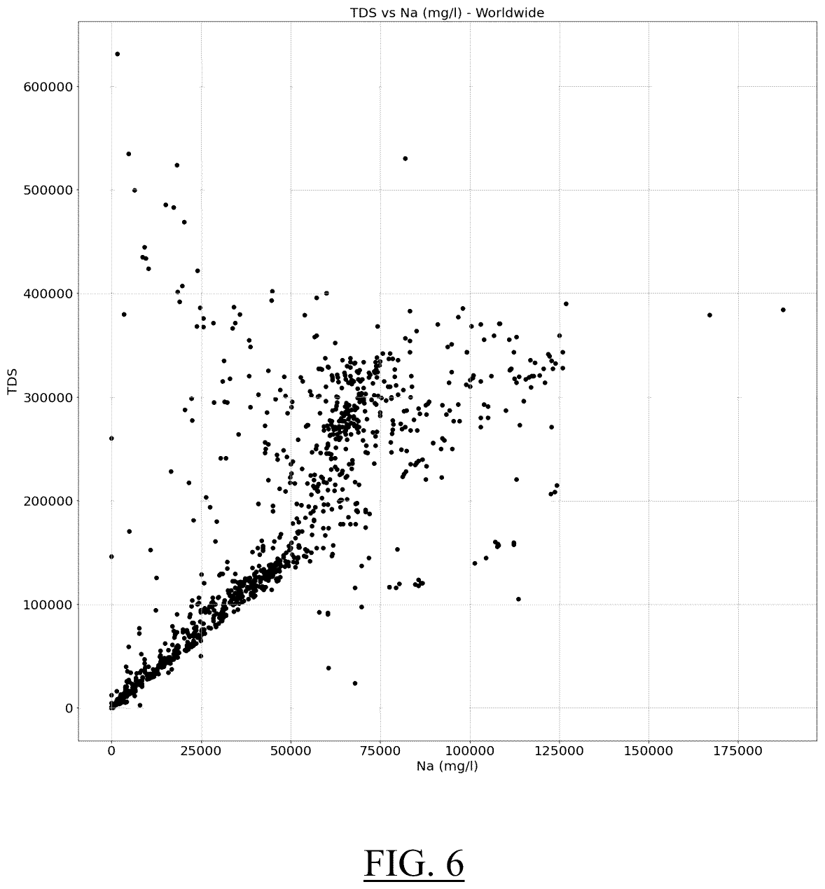

FIG. 5 and FIG. 6 show examples of the relationship between Cl and total dissolved salts (TDS) and between Na and TDS respectively. Both figures display values for a worldwide dataset. In both cases, a strong correlation is observed between the given element and the TDS. These figures show that the chemical elements may present a higher prediction power if the machine-learning is performed basin-wise (compared to worldwide), when the predicted output is a concentration of Lithium, but that this may be untrue for another type of predicted quantity, such as TDS.

1. A computer-implemented method of machine-learning a plurality of predictive basin-wise models each configured for predicting a concentration of an element at a given location in a saline aquifer of a respective basin, the method comprising, for each basin and with respect to a predetermined set of one or more geochemical variables:
providing a dataset comprising, for respective saline aquifer locations of the basin, training samples each including a measurement of one or more geochemical variables of the predetermined set, and a respective ground truth value representing a concentration of the element at the respective saline aquifer location; and
learning the predictive basin-wise model based on the dataset.

2. The method of claim 1, wherein the element is a metal.

3. The method of claim 2, wherein the element is lithium.

4. The method of claim 2, wherein the predetermined set of one or more geochemical variables comprises a concentration of any one or any combination of the following chemical elements: Cl, Ca, Na, B, Mg, Sr, and/or K.

5. The method of claim 1, wherein each predictive basin-wise model comprises an ensemble-learning model.

6. The method of claim 5, wherein the ensemble-learning model is a tree-based model, for example an XG boost model or a Random Forest model.

7. The method of claim 1, wherein each predictive basin-wise model comprises respective alternative sub-models, each sub-model being configured for predicting the concentration of the element at the given location in the saline aquifer when inputted with a measurement of a respective combination of the one or more geochemical variables, each sub-model being learnt on corresponding portions of the training samples of the dataset.

8. The method of claim 7, wherein the dataset comprises missing values, and the learning of each respective sub-model is based on training samples having no missing value in the portion thereof corresponding to the respective sub-model.

9. A method comprising using a machine-learnt predictive basin-wise model configured for predicting a concentration of an element at a given location in a saline aquifer of a respective basin, the method comprising:
providing a given measurement of one or more geochemical variables of a predetermined set of one or more geochemical variables at the given location, and
predicting the concentration of the element at the given location by applying the predictive basin-wise model to the given measurement.

10. The method of claim 9, wherein the one or more geochemical variables of the given measurement form a portion of the predetermined set, and the predicting of the concentration of the element at the given location comprises applying a sub-model of the predictive basin-wise model corresponding to said portion of the predetermined set.

11. The method of claim 9, wherein the method further comprises:
providing a plurality of predictive basin-wise models each configured for predicting a concentration of an element at a given location in a saline aquifer of a respective basin;
providing a value of one or more geographical variables representing a given location;
based on the value of the one or more geographical variables, determining a given basin corresponding to the given location;
selecting the predictive basin-wise model corresponding to the given basin; and
predicting the concentration of the element at the given location by applying the selected predictive basin-wise model to the provided measurement of the one or more geochemical variables.

12. A device comprising a non-transitory computer readable storage medium having recorded thereon a data structure, the data structure comprising at least one of:
i. a plurality of predictive basin-wise models learnt by performing a computer-implemented method of machine-learning a plurality of predictive basin-wise models each configured for predicting a concentration of an element at a given location in a saline aquifer of a respective basin, the method comprising, for each basin and with respect to a predetermined set of one or more geochemical variables:
providing a dataset comprising for respective saline aquifer locations of the basin, training samples each including a measurement of one or more geochemical variables of the predetermined set, and a respective ground truth value representing a concentration of the element at the respective saline aquifer location; and
learning the predictive basin-wise model based on the data,
ii. a computer program comprising instructions for performing a computer-implemented method of machine-learning plurality of predictive basin-wise models each configured for predicting a concentration of an element at a given location in a saline aquifer of a respective basin, the method comprising, for each basin and with respect to a predetermined set of one or more geochemical variables:
providing a dataset comprising, for respective saline aquifer locations of the basin, training samples each including a measurement of one or more geochemical variables of the predetermined set, and a respective ground truth value representing a concentration of the element at the respective saline aquifer location; and
Learning the predictive basin-wise model based on the data, and
iii. a computer program comprising instructions for performing a computer-implemented method of using a machine-learnt predictive basin-wise model configured for predicting a concentration of an element at a given location in a saline aquifer of a respective basin, the method comprising:
providing a given measurement of one or more geochemical variable of a predetermined set of one or more geochemical variables at the given location, and
predicting the concentration of the element at the given location by applying the predictive basin-wise model to the given measurement.

13-17. (canceled)

18. The device of claim 12, wherein the device further comprises a processor coupled to the computer readable storage medium.

19. The device of claim 12, wherein the element is a metal.

20. The device of claim 19, wherein the element is lithium.

21. The device of claim 19, wherein the predetermined set of one or more geochemical variables comprises a concentration of any one or any combination of the following chemical elements: Cl, Ca, Na, B, Mg, Sr, and/or K.

22. The method of claim 9, wherein:
predicting a concentration of an element is performed for one or more first locations of the saline aquifer; and
the method further comprises determining one or more second locations of the saline aquifer for storing CO2 based on the prediction.

23. The method of claim 22, further comprising storing CO2 in at least one second location, the storing including withdrawing water from the at least one second location.

24. The method of claim 23, further comprising recovering the element in the withdrawn water.
